(19) United States
(12) Patent Application Publication
(10) Pub. No.: US 2025/0102387 A1
(43) Pub. Date: Mar. 27, 2025

(54) PRESSURE MEASURING DEVICE

(71) Applicants: Debiotech S.A., Lausanne (CH); Fresenius Medical Care Holdings, Inc, Waltham, MA (US); Fresenius Medical Care Deutschland GmbH, Bad Homburg (DE)

(72) Inventors: William BOVY, Lausanne (CH); Eric CHAPPEL, Lausanne (CH); Pierre THIEBAUD, Lausanne (CH); Phong NGUYEN, Livermore, CA (US); Massimo CAPPELLO, Capergnanica (IT)

(21) Appl. No.: 18/473,954

(22) Filed: Sep. 25, 2023

(51) Int. Cl. G01L 19/00; A61M 5/168; G01L 9/00

(52) U.S. Cl. CPC G01L 19/0023; A61M 5/168; G01L 9/0041

(57) ABSTRACT

The present disclosure relates to a pressure measuring device configured to measure positive pressure and negative pressure. The pressure measuring device may comprise a sealing part, a ring configured to be in contact in use, and a protrusion.

### TECHNICAL FIELD

The present invention relates to a device for measuring the pressure of a fluid, for example a pressure measuring device of a type having a pressure sensor/transducer. Such a pressure measuring device may be used for all kinds of fluids in the medical field, e.g., for pressure determination in fluid circulation device such as those encountered during dialysis.

### BACKGROUND

In the medical field, circulation of a fluid to/from a patient prohibits any risk of contamination of the patient and so contamination of the fluid/liquid. A disposable set of fluid circulation device overcomes this risk but involves some other difficulties. Indeed, the disposable set may be configured for one use only and needs to be economically manufactured to reduce its cost. For control and monitoring purposes, it is desirable to measure the fluid pressure within the disposable set. However, this set cannot comprise any expensive element such as a transducer or other electronic components.

Therefore, such a pressure transducer must be deported on an apparatus configured to be used successively with several disposable sets and the pressure transducer cannot be in direct contact with the fluid. The pressure transducer may be configured to be coupled with the disposable set so as to measure the pressure of the fluid present into the disposable set. In this case, the device may comprise an interface configured to transmit the fluid pressure to the pressure transducer. The interface may comprise a membrane such as a flexible membrane.

In the case of pressure measurement, two cases may be differentiated:
the pressure is higher than a reference value (overpressure) (the reference value may be the atmospheric pressure); or
the pressure is lower than a reference value (under-pressure) (the reference value may be the atmospheric pressure).

In the case of overpressure, a membrane can simply be in contact with the pressure transducer (force or pressure). However, in some cases, the contact of the membrane on the measurement surface of the pressure transducer can induce measurement errors. Therefore, it can be necessary to maintain a fluid tight coupling around the membrane linking the pressure transducer to the disposable set.

And in the case of under-pressure, it is essential to maintain a fluid tight coupling around the membrane linking the pressure transducer to the disposable set, without this fluid tight coupling, the under-pressure cannot be measured by the pressure transducer.

Several problems may be underlined:
Fluid tightness: the coupling between the disposable set and the pressure transducer must be tight to measure the under-pressure and the overpressure.
Resistance to foreign matters: the coupling between the disposable set and the pressure transducer must be tight even in the presence of foreign matter (particles, fibers, hair, . . . )
Deformation of the membrane: to improve the resistance to foreign matters, the person skilled in the art would increase the coupling force (compression ratio) between the disposable set and the pressure transducer. Such a crushing can induce an uncontrolled deformation of the membrane and cause an uncontrolled shift of the measurement.
Flexibility/mobility of the membrane: the membrane should be flexible/movable enough to transmit the pressure without offset or shifting,

Therefore, the sealing between the disposable set and the pressure transducer must be tight enough to measure under-pressure during treatment but the seal cannot be crushed in order to prevent any uncontrolled deformation of the membrane. In case where the membrane is a flexible element, the deformation of the flexible element has to be only caused by the fluid pressure.

Furthermore, the interface of such a medical measurement device requires technical solutions which should be simple, reliable, and replicable. The current technical solutions are not able to fulfil all necessary requirements. There is therefore a need to improve the pressure measurement of fluid in medical fluid circulation devices and to ensure reliability and assembly repeatability.

### SUMMARY

This general description is provided to introduce a selection of disclosures in a simplified form that are further described below in the Detailed Description. This general description is neither intended to necessarily identify key features or essential features of the claimed subject matter, nor is it intended as an aid in determining the scope of the claimed subject matter.

A first aspect of the disclosure relates to a pressure sensor device which comprises a disposable set (e.g., a cartridge) and a pressure transducer. The disposable set may comprise rigid body, a membrane (e.g., a flexible membrane) covering at least a portion of the rigid body, and medical fluid chamber defined by at least one of a wall of the rigid body and a wall of the membrane.

The membrane may be configured to transmit the fluid pressure of the medical fluid present in the disposable set to the pressure transducer. For example, the membrane may comprise a movable part (e.g., a flexible part) configured to transmit positive and negative pressures of the medical fluid to the pressure transducer in use, and a sealing part that may be connected to the movable part. The membrane may further comprise an attachment part that is fixed to the rigid body. In this case, the sealing part may connect the attachment part to the flexible part.

The pressure transducer may be configured to measure the fluid pressure of the medical fluid present in the disposable set. The pressure transducer may comprise a rigid ring that may be configured to be in contact with the sealing part of the membrane in order to create a sealed cavity between the membrane and the pressure transducer in use. The pressure transducer may further comprise a protrusion configured for example to add compression against the sealing part so that the seal is maintained even in the presence of foreign matter on the rigid ring. The protrusion may be arranged on the rigid ring (e.g., more rigid than the sealing part of the membrane).

The sealing part may comprise at least one of a first contact surface configured to be in contact with the rigid body and a second contact surface configured to be in contact with the rigid ring in use. The first contact surface may be opposite the second surface contact. The sealing part may be configured to be sandwiched between the rigid body and the rigid ring in use. The sealing part may be configured to be maintained in its position by the rigid body when the rigid ring is pressed against the sealing part. The sealing part, the flexible part and the attachment part may comprise the same material.

The rigid ring may comprise a surface substantially parallel to at least one of the first and the second contact surface of the sealing part in use. The protrusion may be arranged in the middle of the planar surface of the rigid ring. The protrusion may be arranged along the entire length of surface of the ring in contact with the sealing part in use.

The protrusion may comprise a triangular shape having a free end/peak configured to deform the sealing part when the rigid ring is pressed against the sealing part.

The sealing part may be configured to be flat when the disposable is not coupled to the pressure transducer and to fit the protrusion (e.g., by its compliance) and contact the planar surface of the ring when the disposable set is coupled to the pressure transducer.

The disposable set may comprise a cartridge and may be discarded after its single use, while the pressure transducer may be arranged in re-usable machine. The re-usable machine may be configured to be successively used with several disposable sets.

In one embodiment, the width of the sealing part in contact with the rigid ring may be equal or greater than the width of the rigid ring. In another embodiment, the width of the rigid ring in contact with the sealing part may be equal or greater than the width of the sealing part.

The sealing part may comprise a planar contact surface (e.g., the second contact surface) configured to be in contact with the rigid ring in use. The sealing part may further comprise a sealing protrusion configured to be in contact with the rigid ring in use or the rigid body. The cross-section of the sealing protrusion may comprise a square, triangular, or rounded shape.

A second aspect of the disclosure relates to a fluid pressure measuring unit for medical device which comprises a disposable set and a pressure transducer.

The disposable set may comprise at least one of a rigid body, a membrane, and a medical fluid chamber defined by rigid walls of the rigid body and at least a part of the membrane. The membrane may comprise at least one of a flexible part, an attachment part, and a sealing part. The flexible part may be configured to cover at least partially the medical fluid chamber, the attachment part may be fixed to the rigid body, and the sealing part may connect the attachment part to the flexible part.

The pressure transducer may comprise a measuring surface and a rigid ring extending away from the measuring surface.

The membrane may be configured to transmit the fluid pressure of the medical fluid present in the disposable set to the pressure transducer. The pressure transducer may be configured to measure the fluid pressure of the medical fluid present in the disposable set.

The sealing part and the rigid ring may be configured to be mechanically contacting each other in use. The movable part of the membrane and the measuring surface may be configured to be separated by a fluid tight chamber in use. The fluid pressure measuring unit may further comprise a sealing protrusion configured to insure a fluid tight sealing of the fluid tight chamber even in the presence of foreign matter on the rigid ring.

The sealing part may be configured to be sandwiched between the rigid body and the rigid ring in use.

The sealing protrusion may be arranged on a first contact surface of the sealing part of the membrane and is configured to be in contact with a surface of the rigid body. The sealing protrusion may be arranged on a second contact surface of the sealing part of the membrane and may be configured to be in contact with a surface of the rigid ring in use. The rigid ring may further comprise a groove configured to receive the sealing protrusion of the sealing part in use.

The sealing protrusion may be arranged on a contact surface of rigid ring and may be configured to be in contact with a surface of the sealing part in use. The sealing protrusion may be arranged at the middle of the contact surface of the rigid ring, on an inner edge of the contact surface of the rigid ring, and/or on an outer edge of the contact surface of the rigid ring. The sealing protrusion may extend at its base from the contact surface of the rigid ring in a direction along a perpendicular axis of the measuring surface. The width of the base of the sealing protrusion may be at least half the width of the contact surface of the rigid ring. The width of the base of the sealing protrusion may be at least less than half the width of the contact surface of the rigid ring.

The sealing protrusion may comprise a rounded shaped end or a sharped end. The flexible part may comprise at least one of a circular shape, a corrugated shape, and concentric waves.

The flexible part and the measuring surface may be configured to be substantially parallel in use.

### BRIEF DESCRIPTION OF DRAWINGS

The present disclosure will be better understood at the light of the following detailed description which contains non-limiting examples illustrated by the following figures.

FIG. 1 illustrates an embodiment of a medical system.

FIGS. 2a and 2b show an example of a disposable set and a pressure transducer.

FIGS. 3a and 3b show a cross-sectional view of an embodiment comprising a disposable set and a pressure transducer but without protrusion.

FIG. 4 shows an example of membrane too compressed against the pressure transducer.

FIG. 5 shows a cross-sectional view of a possible embodiment comprising a protrusion arranged on the ring.

FIGS. 6a and 6b show cross-sectional views of a possible embodiment comprising a protrusion arranged on the ring.

FIG. 7 shows a cross-sectional view and a zoom of a membrane coupled to a ring having a protrusion.

FIGS. 8 and 9 show 3d views of a pressure transducer with different shapes of the protrusion.

FIGS. 10 and 11 show cross-sectional views of a possible embodiment comprising a protrusion arranged on the membrane.

FIG. 12 shows a cross-sectional view of an embodiment wherein the ring comprises a groove.

FIG. 13 shows a cross-sectional view of different protrusion shapes.

### LIST OF ELEMENTS

1 Pressure measuring device
2 Disposable set/cartridge
3 Pressure transducer
4 Membrane
5 Medical fluid chamber
6 Rigid body
7 Attachment part
8 Movable part
9 Sealing part
10 Ring
11 Measuring surface
12 Cavity
13 Opening
14 Protrusion
15 Groove
100 Medical system
101 Disposable part/set
102 Reusable part/apparatus
104 Bag (Drain bag)
105 Bag (Fresh solution)
106 Cartridge
110 Processor
111 Screen (device for example tablet with a touch screen)
112 Other element such as button
113 Sensor/Pressure transducer
114 Actuator
115, 116 Other elements

### DETAILED DESCRIPTION

The following detailed description includes references to the accompanying drawings, which form a part of the detailed description. The drawings show, by way of illustration, specific embodiments in which the disclosure may be practiced. These embodiments, which are also referred to herein as “examples,” are described in enough detail to enable those skilled in the art to practice the disclosure. The embodiments may be combined, other embodiments may be utilized, or structural, logical and electrical changes may be made without departing from the scope of the present disclosure. The following detailed description is, therefore, not to be taken in a limiting sense, and the scope of the present disclosure is defined by the appended claims and their equivalents.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the claims, the singular forms “a”, “an”, and “the” encompass embodiments having plural referents, unless the content clearly dictates otherwise.

As used in this specification and the claims, any direction referred to herein, such as “top”, “bottom”, “left”, “right”, “upper”, “lower”, and other directions or orientations are described herein for clarity in reference to the figures and are not intended to be limiting of an actual device or system unless the content clearly dictates otherwise. Devices and systems described herein may be used in several directions and orientations.

As used in this specification and the claims, “have”, “having”, “include”, “including”, “comprise”, “comprising” or the like are used in their open-ended sense, and generally mean “including, but not limited to”.

As used in this specification and the claims, the term “or” is generally employed in its sense including “and/or” unless the content clearly dictates otherwise.

As used in this specification and the claims, “at least one of A, B, and C”, “at least one of A, B or C”, “selected from the group consisting of A, B, C, and combinations thereof” or the like are used in their open ended sense including “only A, or only B, or only C, or any combination of A, B and C” unless the content clearly dictates otherwise.

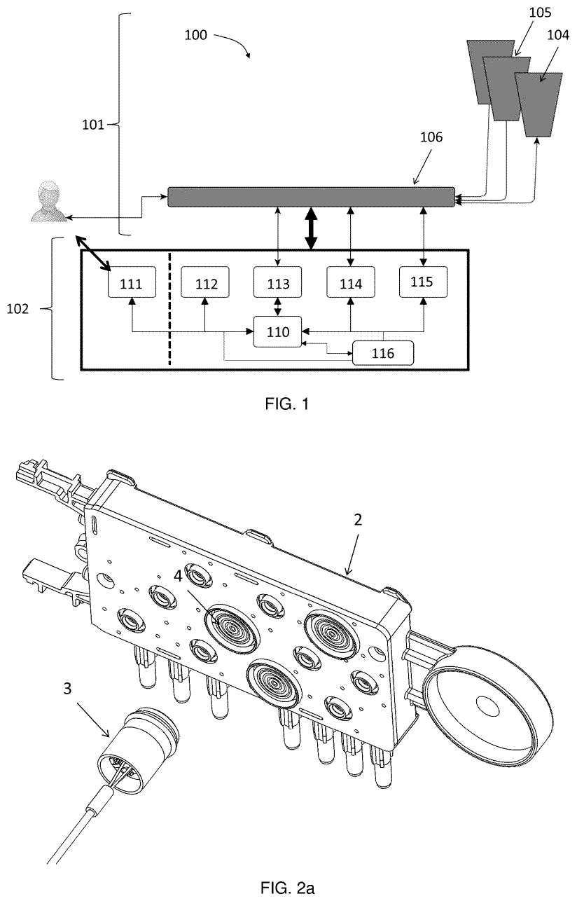

According to various embodiments as shown by FIG. 1, the medical system (100) may include at least one of a reusable part (102) (also called apparatus) and a disposable part/set (101). The disposable part may include the elements which must be discarded after a predetermined number of uses, for example, after a single use. The working life of the disposable part may directly depend on the number of treatments. These elements may be the elements which have been wetted by a solution for example a dialysate solution or blood, for example, a part of a fluid circuit. The disposable part may comprise at least one of a tube, a connector, a port, a cartridge, a valve, . . .

The reusable part may comprise the expensive elements for example sensor, electronic part, screen, actuator of the valve or of pump, processor, memory, . . . . The reusable part may be successively used with several disposable parts. The reusable part may comprise elements which may be replaced when the components are too worn, become broken or after a predetermined period of time, but much longer than a single treatment. The change of the reusable part may depend on the component wear.

The disposable part (101) may comprise at least one of a bag (104, 105), a tube and a cartridge (106). The reusable part (102) may comprise at least one of one or more processors (110), one or more screens (111), other elements (112) such as one or more buttons, one or more sensors (113), one or more actuators (114) and other elements (115, 16).

According to various embodiments, the reusable part may include a housing in which is arranged components for controlling a medical treatment. For example: a processor, a valve actuator, a sensor, at last a part of a pumping device.

The screen (111) may be touch-screen and may be removably coupled to the housing comprising the other elements of the reusable part (102). All or a part of the elements (111, 112, 113, 114, 115, 116) may be connected or operatively coupled to one or more processors in order to control or monitor the treatment. The processor (110) may execute computer-executable instructions stored in a memory of the system. Some elements of the reusable part (113, 114, 115) may be adapted and intended to be operatively (and/or removably) coupled to the disposable part (101), for example the cartridge (106). The elements (110, 111, 112, 113, 114, 115, 116) may be arranged into the housing of the reusable part.

According to various embodiments, the medical system may comprise a cartridge (106) which define at least a part of the fluidic circuit. The cartridge is preferentially a part of the disposable set. The cartridge/disposable set may include at least one of a rigid frame or body, one or more port, a fluid cavity, one or more membrane (e.g., flexible membrane) adapted to cover the fluid cavity. The membrane may comprise a coupling area adapted to be operatively coupled with a valve actuator (or occluding device) and/or a measurement area adapted to be operatively coupled with a sensor of the reusable part of the system.

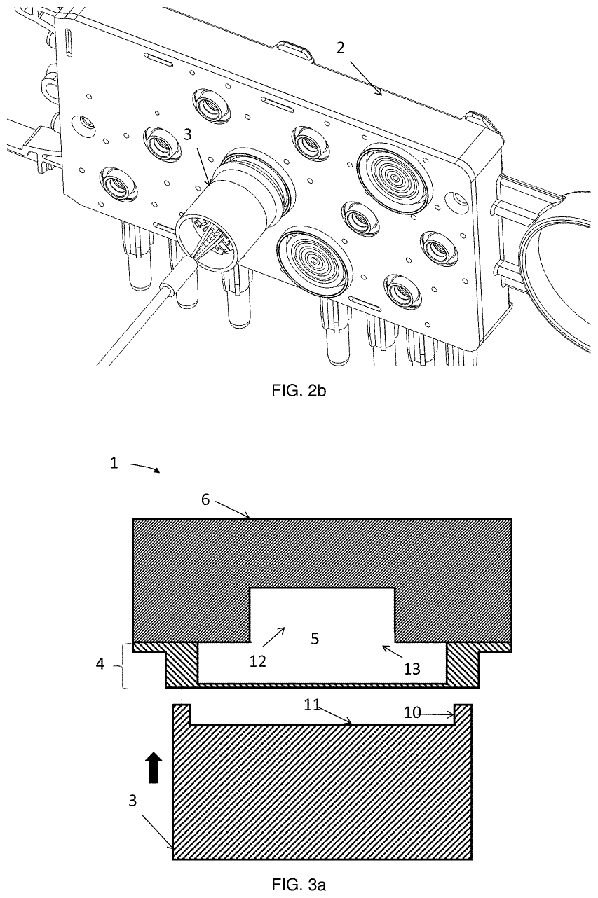

Referring to FIGS. 2a and 2b, these show an example embodiment of a disposable set (2) and a pressure transducer (3). The disposable set (2) comprises at least one medical fluid chamber (not visible here) which is covered by the membrane (4). FIG. 2a shows the disposable set (2) spaced from the pressure transducer (3). FIG. 2b shows the pressure transducer operatively coupled to the disposable set (2). The pressure transducer may be arranged into a housing (not showed here) of the apparatus (as disclosed above). The pressure transducer is configured to be removably coupled to the cartridge/disposable set.

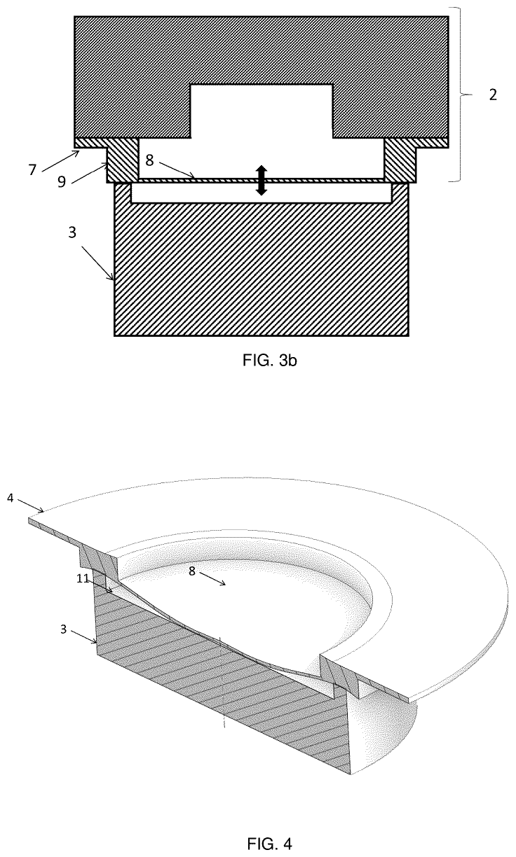

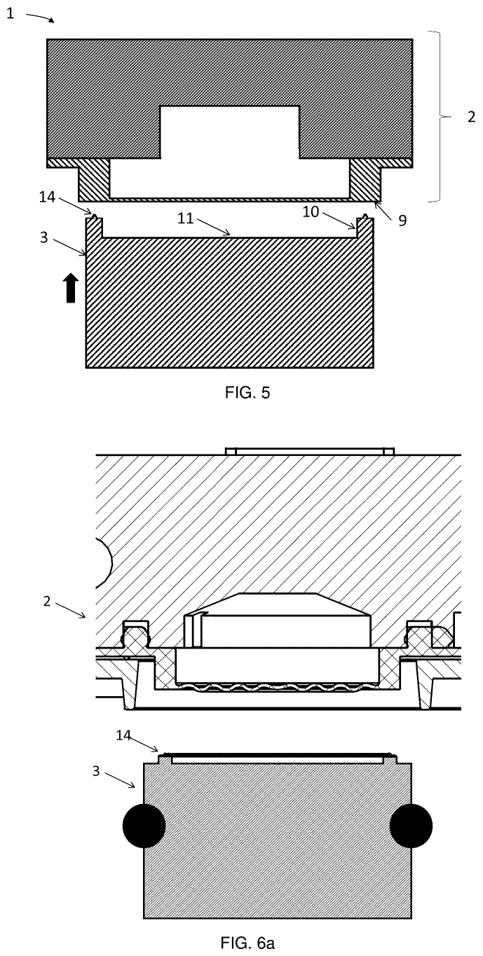

Referring to FIGS. 3a, 3b, and 4, these embodiments do not comprise any sealing protrusion (also called protrusion). These figures show an embodiment comprising a pressure transducer (3) and a disposable set (2). The disposable set may comprise at least one of a rigid body (6) and a membrane (4). The disposable set may further comprise a medical fluid chamber (5) defined by at least of one wall of the rigid body and one wall of the membrane. The rigid body (6) may further comprise at least one of a cavity (12) and an opening (13) which may be covered by the membrane (4). The membrane may comprise at least one of an attachment part (7), a movable part (8) (which may be flexible), and a sealing part (9).

The movable part may be configured to transmit the fluid pressure to the pressure transducer in use (via for example the air trapped into the fluid tight chamber). For example, the movable part may be configured to move (for example bend/extend/stretch) toward and away from the pressure transducer (3) in use. At least one of the movable part, the sealing part and the membrane may comprise an elastic behavior. The membrane may be molded.

The membrane material may comprise at least one of a substantially incompressible material, elastomeric material, silicon, rubber, Polypropylene (PP), Polyethylene (PE), Ethylene Vinyl Alcohol (EVOH), Polyamide (PA), Polychlorotrifluoroethylene (PCTFE), Cyclic Olefin Copolymer (COC), Polycarbonate (PC), Ethylene Vynil Acetate (EVA), Polyvinyl Chloride (PVC), Polyvinylidene Chloride (PVDC), Polystyrene (PS), Polyethylene Terephthalate (PET), Thermoplastic Elastomer (TPE), Thermoplastic polyurethane (TPU), and Polymethacrylate (PMMA/MABS/MBS).

The pressure transducer may comprise at least one of a ring (e.g., a rigid ring) (10) and a measuring surface (11) (which may be connected or operatively coupled to an electronic component connected to the processor of the reusable apparatus). The ring may extend away from the measuring surface of the pressure transducer. The ring may be a part of the body/housing of the pressure transducer. The ring may be rigidly fixed to the pressure transducer.

The ring material may comprise at least one of an incompressible material, a rigid material, metal (such as iron, steel, zinc, chrome, silver, platinum, gold, titanium, copper, nickel, aluminum), a polymer (such as Polypropylene (PP), Cyclic Olefin Copolymer (COC), Polymethacrylate (PMMA/MABS/MBS), Copolyester (PCTG), Polyethylene Terephthalate (PET), Polyether ether ketone (PEEK), Polyamide-imide (PAI), Polyphenylene sulfide (PPS), Polysulfone (PSU), Polybutylene terephthalate (PBT), Acetal, Acrylic, Nylon, ABS, Fluoropolymer, Polytetrafluoroethylene (PTFE), High-density polyethylene (HDPE), Perfluoroalkoxy alkanes (PFA), Polyfluorure de vinylidène (PVDF), or Polycarbonate (PC)), and an alloy.

The ring of the pressure transducer and the sealing part are preferentially aligned in use so that the coupling forms a sandwich comprising the rigid body, the sealing part, and the ring. In this case, the ring is intended/configured to compress the sealing part against the rigid body in use. A fluid tight chamber may be defined by at least one of a wall of the measuring surface, a wall of the ring and a wall of the membrane in use.

In the case of a high compression ratio (more than 90%), the membrane (e.g., the movable part) can be deformed and cause measurement errors due to the contact of the movable part against the measuring surface of the pressure transducer, as shown in FIG. 4. Therefore, the pressure measuring device may be configured to seal with a compression ratio of the sealing part below 90%.

In order to improve the sealing, the pressure measuring device may comprise a protrusion. The protrusion may be configured to provide a seal even if foreign objects are on the ring (or between the ring and the sealing part). Additionally, with the protrusion, the average compression ratio may be reduced to 1-60% (e.g., 20-40%) while still providing a seal. The protrusion may be configured to locally exert a higher compression ratio.

Referring to FIGS. 5 to 13, the description below presents an example solution where the embodiments comprise a protrusion. To avoid unnecessary repetition, the embodiments described below may include any or all of the features of the embodiments described above. Each embodiment described below may further comprise a protrusion.

Referring to FIGS. 5 to 9, in these embodiments, the pressure transducer (3) may comprise a protrusion (14) (also called sealing protrusion). The pressure measuring device may comprise a disposable set (2) and a pressure transducer (3). The pressure transducer (3) may comprise a ring (10) which may extend away from the measuring surface (11). The ring may comprise a base rigidly fixed to the pressure transducer (3).

The ring may comprise a free end on which the protrusion (14) may be arranged. The protrusion may be configured to extend toward the disposable set/sealing part (in use).

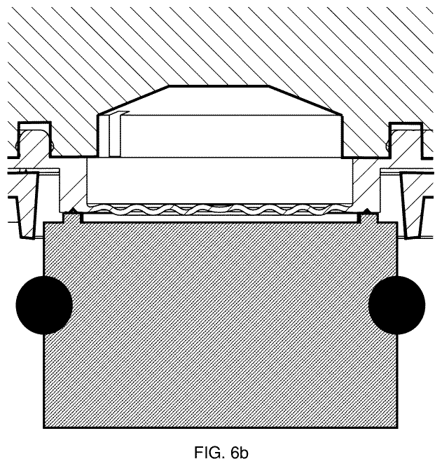

The free end of the ring may comprise a contact surface which may be configured/intended to be in contact with a contact surface of the sealing part (9) of the membrane in use. The contact surface of the ring may be substantially flat in shape and the protrusion may be arranged on the contact surface of the ring. The protrusion may extend (e.g., continuously and/or circumferentially) over the contact surface of the ring (FIGS. 6a, 8 and 9). The ring and the protrusion may be made in a single piece.

The contact surface of the ring and the contact surface of the sealing part may be substantially parallel in use.

FIG. 6a shows a possible embodiment where the disposable set and the pressure transducer are not coupled while FIG. 6b shows the same embodiment but with the disposable set (2) coupled (use position) to the pressure transducer (3).

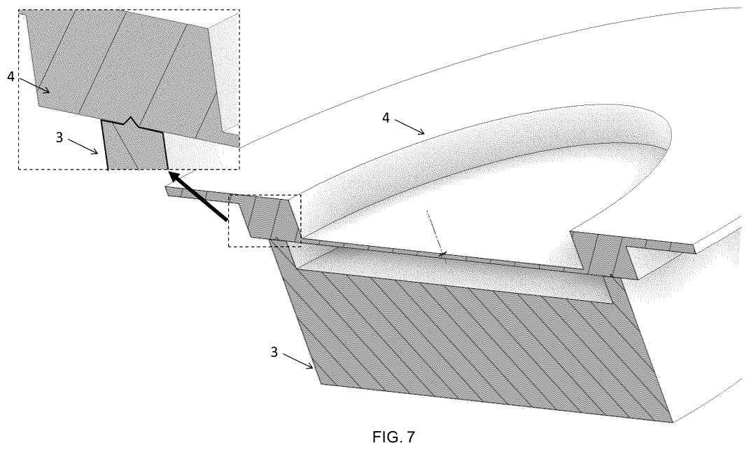

Referring to FIGS. 6b and 7, the contact surface of the sealing part may be substantially flat in shape but may be configured to fit the protrusion when the ring is compressed against the sealing part (e.g., in use). In this case, the sealing part of the membrane is sandwiched between surface of the rigid body of the disposable set and the contact surface of the ring having the protrusion and the sealing part fits over the protrusion of the ring (when the pressure transducer is coupled to the disposable set).

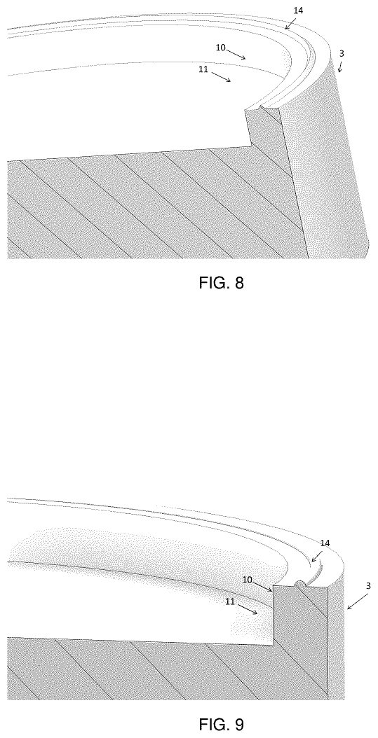

FIGS. 8 and 9 disclose two different shapes of the protrusion (14) arranged on the ring (10), FIG. 13 shows other possible shapes of the protrusion, but the shape of the protrusion should not be limited to these examples.

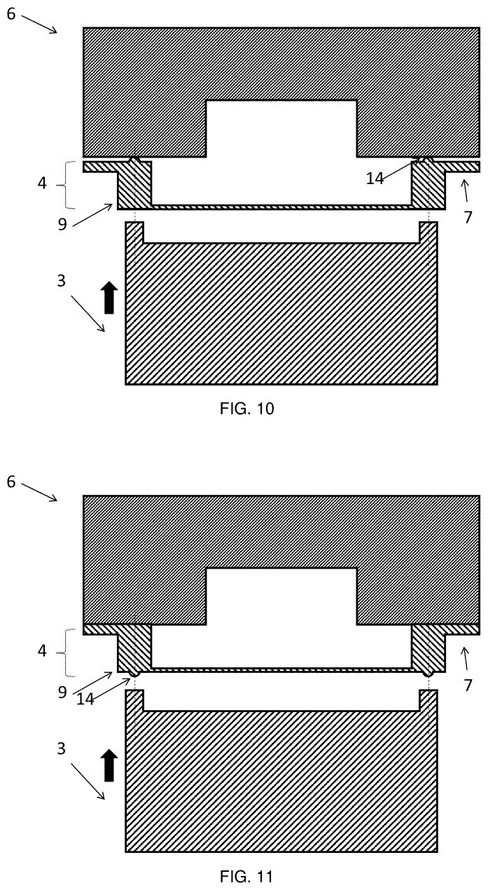

FIGS. 10, 11, and 12 disclose embodiments wherein the protrusion may be arranged on the membrane. These embodiments may further comprise a protrusion on the ring, but this is not shown.

Referring to FIG. 10, the membrane (4) may comprise a protrusion (14) arranged on a surface in contact with the rigid body of the disposable set. The sealing part of the membrane may comprise a first contact surface configured to be in contact with the rigid body (6) and on which the protrusion may be arranged. The protrusion may extend (e.g., continuously and/or circumferentially) over the first contact surface of the sealing part of the membrane.

Referring to FIG. 11, the membrane (4) may comprise a protrusion (14) arranged on a surface configured to be in contact with the ring in use. The sealing part of the membrane may comprise a second contact surface configured to be in contact with the ring in use and on which the protrusion may be arranged. The protrusion may extend (e.g., continuously and/or circumferentially) over the second contact surface of the sealing part of the membrane.

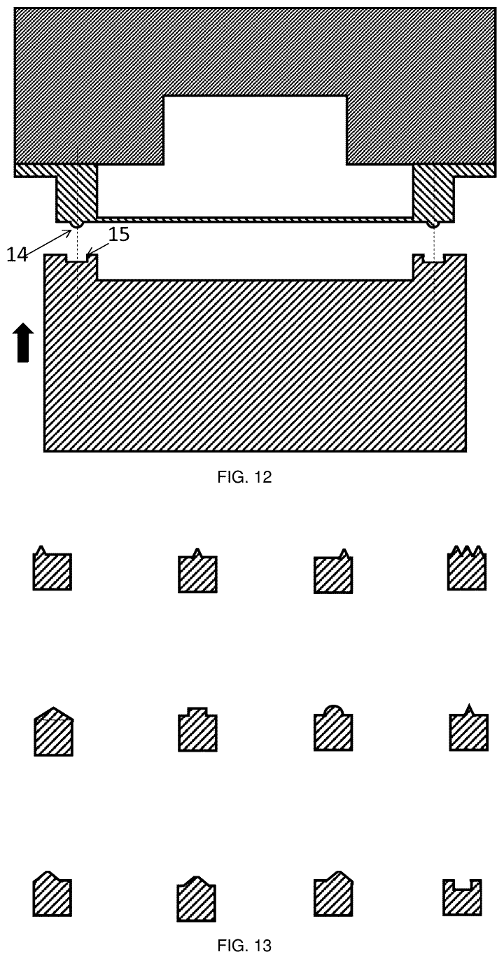

Referring to FIG. 12, the ring may comprise a groove (15) configured to receive the sealing protrusion of the sealing part in use.

Referring to FIG. 13, the protrusion arranged on the ring or on the membrane may comprise several shapes. The protrusion may by be arranged in the middle of the contact surface, on its outer edge and/or on its inner edge. The cross-section of the protrusion may have a square, round, or triangular shape. The width of the base of the protrusion may be equal to or less than the width of its support (ring or sealing part).

The movable part (8) of the membrane (4) may have a part or be completely corrugated in order to facilitate its movement. Furthermore, this corrugated shape may be configured to reduce the stress in the membrane when the pressure transducer is press against the membrane. Indeed, when the compression ratio is too high, the corrugated shape may attenuate the stress in the membrane and thus allow a measurement closer to reality.

1. A pressure sensor device comprising
A cartridge comprising:
A rigid body,
A membrane covering at least a portion of the rigid body, and
A medical fluid chamber defined by at least one of a wall of the rigid body and a wall of the membrane, and
A pressure transducer configured to measure the fluid pressure of the medical fluid,
wherein the membrane comprises:
a movable part configured to transmit positive and negative pressures of the medical fluid to the pressure transducer in use, and
a sealing part connected to the movable part,
wherein the pressure transducer comprises a rigid ring configured to be in contact with the sealing part of the membrane in order to create a sealed cavity between the membrane and the pressure transducer in use and wherein the rigid ring comprises a protrusion configured so that the seal is maintained even in the presence of foreign matter on the ring.

2. Pressure sensor device according to the claim 1, wherein the sealing part comprises a first contact surface configured to be in contact with the rigid body.

3. Pressure sensor device according to claim 1, wherein the sealing part comprises a second contact surface configured to be in contact with the rigid ring in use.

4. Pressure sensor device according to claim 2, wherein the rigid ring comprises a surface substantially parallel to at least one of the first and the second contact surface of the sealing part in use.

5. Pressure sensor device according to claim 1, wherein the sealing part is configured to be sandwiched between the rigid body and the rigid ring in use.

6. Pressure sensor device according to claim 1, wherein the sealing part is maintained in its position by the rigid body when the rigid ring is pressed against the sealing part.

7. Pressure sensor device according to claim 1, wherein the protrusion is arranged substantially in the middle of the planar surface of the rigid ring.

8. Pressure sensor device according to claim 1, wherein the protrusion has a triangle shape having a free peak configured to deform the sealing part when the rigid ring is pressed against the sealing part.

9. Pressure sensor according to claim 1, wherein the protrusion is arranged along the entire length of surface of the ring in contact with the sealing part in use.

10. Pressure sensor device according to claim 1, wherein the sealing part, the movable part and the attachment part comprise the same material.

11. Pressure sensor device according to claim 1, wherein the cartridge is a disposable device, and the pressure transducer is arranged in re-usable machine.

12. Pressure sensor device according to claim 11, wherein the re-usable machine is configured to be successively used with several cartridges while the cartridge has to be discarded after its single use.

13. Pressure sensor device according to claim 1, wherein the width of the sealing part in contact with the rigid ring is greater than the width of the rigid ring.

14. Pressure sensor device according to claim 1, wherein the width of the rigid ring in contact with the sealing part is greater than the width of the sealing part.

15. Pressure sensor device according to claim 1, wherein the sealing part comprises a planar contact surface configured to be in contact with the rigid ring in use.

16. Pressure sensor device according to claim 1, wherein the sealing part comprises an additional protrusion configured to be in contact with the rigid ring in use.

17. Pressure sensor device according to claim 16, wherein the additional protrusion comprises a rounded shape.

18. Fluid pressure measuring unit for medical device comprising:
a disposable set having a rigid body, a membrane, and a medical fluid chamber defined by rigid walls of the rigid body and at least a part of the membrane, wherein the membrane comprises:
a movable part configured to cover at least partially the medical fluid chamber, and
a sealing part connected to the movable part; and
a pressure transducer having a measuring surface and a rigid ring extending away from the measuring surface,
wherein the sealing part and the rigid ring are configured to be mechanically contacting each other and the movable part and the measuring surface are configured to be separated by a fluid tight chamber in use and wherein the fluid pressure measuring unit further comprises a sealing protrusion configured to insure a fluid tight sealing of the fluid tight chamber even in the presence of foreign matter on the rigid ring.

19. Fluid pressure measuring unit according to claim 18, wherein the sealing part is configured to be sandwiched between the rigid body and the rigid ring in use.

20. Fluid pressure measuring unit according to claim 18, wherein the sealing protrusion is arranged on a first contact surface of the sealing part of the membrane and is configured to be in contact with a surface of the rigid body.

21. Fluid pressure measuring unit according to claim 18, wherein the sealing protrusion is arranged on a second contact surface of the sealing part of the membrane and is configured to be in contact with a surface of the rigid ring in use.

22. Fluid pressure measuring unit according to claim 21, wherein the rigid ring comprises a groove configured to receive the sealing protrusion of the sealing part in use.

23. Fluid pressure measuring unit according to claim 18, wherein the sealing protrusion is arranged on a contact surface of rigid ring and is configured to be in contact with a surface of the sealing part in use.

24. Fluid pressure measuring unit according to claim 23, wherein the sealing protrusion is arranged at the middle of the contact surface of the rigid ring.

25. Fluid pressure measuring unit according to claim 23, wherein the sealing protrusion is arranged on an inner edge of the contact surface of the rigid ring.

26. Fluid pressure measuring unit according to claim 23, wherein the sealing protrusion is arranged on an outer edge of the contact surface of the rigid ring.

27. Fluid pressure measuring unit according to claim 23, wherein the sealing protrusion extends at its base from the contact surface of the rigid ring in a direction along a perpendicular axis of the measuring surface.

28. Fluid pressure measuring unit according to claim 27, wherein the width of the base of the sealing protrusion is at least half the width of the contact surface of the rigid ring.

29. Fluid pressure measuring unit of claim 27, wherein the width of the base of the sealing protrusion is at least less than half the width of the contact surface of the rigid ring.

30. Fluid pressure measuring unit according to claim 18, wherein the sealing protrusion comprises a rounded shaped end or a sharped end.

31. Fluid pressure measuring unit according to claim 18, wherein the movable part comprises a circular shape.

32. Fluid pressure measuring unit according to claim 18, wherein movable part comprises a corrugated shape.

33. Fluid pressure measuring unit according to claim 31, wherein the movable part comprises concentric waves.

34. Fluid pressure measuring unit according to claim 18, wherein the movable part and the measuring surface are configured to be substantially parallel in use.
